(19) United States
(12) Patent Application Publication
(10) Pub. No.: US 2017/0239410 A1
(43) Pub. Date: Aug. 24, 2017

(54) INFUSATE FRAME

(71) Applicant: Medtronic, Inc., Minneapolis, MN (US)

(72) Inventors: David B. Lura, Maple Grove, MN (US); Martin T. Gerber, Maple Grove, MN (US); Christopher M. Hobot, Rogers, MN (US); Thomas E. Meyer, Stillwater, MN (US); Jin Huang, Shanghai (CN)

(21) Appl. No.: 15/588,772

(22) Filed: May 8, 2017

Related U.S. Application Data
(63) Continuation-in-part of application No. 15/219,238, filed on Jul. 25, 2016.
(60) Provisional application No. 62/196,891, filed on Jul. 24, 2015.

(51) Int. Cl. A61M 1/16; A61K 33/00; A61M 1/34

(52) U.S. Cl. CPC A61M 1/1668; A61M 1/1666; A61M 1/3462; A61K 33/00

(57) ABSTRACT

The invention relates to an infusate frame for use in dialysis. The infusate frame includes one or more openings cut-out of a rigid support frame. The one or more openings are sized and shaped complementary to one or more infusate containers, ensuring that only a single infusate container can occupy a specific opening.

### CROSS-REFERENCE TO RELATED APPLICATIONS

This application is a continuation-in-part of U.S. patent application Ser. No. 15/219,238, filed on Jul. 25, 2016, which claims the benefit of U.S. Provisional Patent Application No. 62/196,891, filed on Jul. 24, 2015, the entire contents of which is incorporated herein by reference.

### FIELD OF THE INVENTION

The invention relates to and infusate frame for use in dialysis. The infusate frame includes one or more openings cut-out of a rigid support frame. The one or more openings are sized and shaped complementary to one or more infusate containers, ensuring that only a single infusate container can occupy a specific opening.

### BACKGROUND

During priming of a dialysis system and during dialysis treatment, specific and concentrations of specific solutions, such as sodium chloride, sodium bicarbonate, and cation infusates, must be added to the dialysate flow path. Further, many cations, such as potassium, calcium and magnesium, can cross the dialyzer and be removed from a patient during dialysis. The cations must be added back into the dialysate to maintain the concentration of the cations at a desired level. Sodium bicarbonate can be used during dialysis as a buffer to control the pH of the dialysate and to treat acidosis by delivering bicarbonate across the dialysis membrane to the patient receiving a treatment. The amounts of sodium chloride, sodium bicarbonate and other cations added to dialysate should be closely monitored and controlled. Further, the amounts of each of the solutions necessary can vary considerably.

There is a need for ensuring proper solutes are added in proper amounts to the dialysate. To facilitate use of dialysis by personnel, systems and methods are needed that can ensure that any of the solutes or solutions are properly added to the dialysis system. Further, systems and methods are needed to ensure that all necessary components to be used during dialysis are connected to the dialysis system at the correct locations for a dialysate flow path. There is a further need for a system that can allow users of varying skill levels to easily configure the dialysis system for disinfection, and ensure that the dialysis system can be used outside of a clinical setting, such as in a patient's home, is needed.

### SUMMARY OF THE INVENTION

The first aspect of the invention relates to an infusate frame. In any embodiment, the infusate frame can include a rigid planar surface having at least a first lip disposed at a periphery of the planar surface and extending outwardly from the planar surface; the lip complementary to a ledge disposed on a dialysis machine; one or more openings cut-out on the planar surface; the one or more openings extending from a first side to a second side of the planar surface; the one or more openings having a size and/or shape complementary to one or more infusate container; and a vertical axis of the opening can be aligned to a fluid connector disposed on the dialysis machine.

In any embodiment, the one or more openings can be tapered inwardly.

In any embodiment, at least one opening can be slotted to a periphery of the planar surface.

In any embodiment, the one or more openings can be selected from a circular shape, a square shape, a triangular shape, or an oval shape.

In any embodiment, the infusate frame can have one or more handles disposed on a periphery of the planar surface; the one or more handles extending upwardly from the planar surface.

In any embodiment, the infusate container can be selected from the group of a sodium bicarbonate container, a sodium chloride container, and a cation infusate container.

In any embodiment, the infusate frame can have at least one vertical support disposed on a periphery of the planar surface; the vertical support extending upwardly from the periphery of the planar surface with a lip extending horizontally from the vertical support.

In any embodiment, at least two openings can be cut-out on the planar surface, the at least two openings having different sizes or shapes.

In any embodiment, the infusate frame can have a second lip opposedly positioned on the planar surface to the first lip.

Any of the features disclosed as being part of the first aspect of the invention can be included in the first aspect of the invention, either alone or in combination.

The second aspect of the invention is drawn to a dialysis system. In any embodiment, the dialysis system can include the infusate frame of the first aspect of the invention having one or more infusate containers containing one or more solute; and a dialysis machine having (i) a dialysate flow path; (ii) a receiving compartment for the infusate frame; and (iii) one or more fluid connectors fluidly connectable to the one or more infusate containers and the dialysate flow path.

In any embodiment, the dialysis system can include one or more pumps and one or more valves fluidly connectable to the one or more fluid connectors; the one or more pumps and one or more valves controlling fluid flow from the one or more infusate containers into the one or more fluid connectors.

In any embodiment, the dialysis system can include a locking mechanism preventing the infusate frame from moving after insertion into the receiving compartment when the locking mechanism is in a locked state.

In any embodiment, the receiving compartment can be sized or shaped complementary to a size or shape of the infusate frame.

In any embodiment, the infusate frame can be formed integrally with the dialysis machine.

In any embodiment, the infusate frame can be removable from the dialysis machine.

In any embodiment, at least one pump can be a bidirectional pump.

In any embodiment, at least one fluid connector can be disposed on a moveable paddle.

In any embodiment, the one or more infusate containers can include a cation infusate container, a sodium chloride container, a sodium bicarbonate container, or combinations thereof.

In any embodiment, the sodium chloride container and sodium bicarbonate container can be fluidly connectable to a valve; wherein the valve is fluidly connected to the dialysate flow path upstream of a sorbent cartridge and downstream of the sorbent cartridge.

Any of the features disclosed as being part of the second aspect of the invention can be included in the second aspect of the invention, either alone or in combination.

### BRIEF DESCRIPTION OF THE DRAWINGS

FIGS. 1A-D show an infusate frame with three openings cut-out of a rigid support frame.

FIGS. 2A-E show an infusate frame having an opening slotted to a periphery of the infusate frame.

FIG. 3 shows a receiving compartment of a dialysis machine for receiving an infusate frame.

FIG. 4 shows a flow diagram of infusate containers.

FIG. 5 shows a partial dialysate flow path and connected infusate containers.

### DETAILED DESCRIPTION OF THE INVENTION

Unless defined otherwise, all technical and scientific terms used herein generally have the same meaning as commonly understood by one of ordinary skill in the relevant art.

The articles “a” and “an” are used to refer to one or to over one (i.e., to at least one) of the grammatical object of the article. For example, “an element” means one element or over one element.

The term “aligned” refers to a configuration of components wherein the components are positioned to a particular arrangement of components. For example, a fluid connection can be aligned to a movable connector to form a fluid connection between the components upon being positioned to a proper alignment.

The term “bi-directional connector” refers to a fluid connector through which fluid can move into or out of a container. The bi-directional connector can include separate channels for fluid movement in either direction, or a single channel used for both adding and removing fluid from the container.

The term “bi-directional pump” refers to a device configured to perform work on a fluid to cause the fluid to flow alternatively in either of two opposing directions.

The term “cation infusate container” refers to a source from which cations can be obtained. Examples of cations include, but are not limited to, calcium, magnesium and potassium. The source can be a solution containing cations or dry compositions that are hydrated by the system. The cation infusate container is not limited to cations and may optionally include other substances to be infused into a dialysate or replacement fluid; non-limiting examples can include glucose, dextrose, acetic acid and citric acid.

The term “circular” refers to a two-dimensional shape generally round, disk shaped, ring-shaped or annular, and having the form of a circle.

The term “complementary,” as used to describe fitting features, refers to one or more fitting features on a first component that are designed to pair or mate with one or more fitting features on a second component. For example, a first component may have a receiving compartment of particular dimensions, and the second component may be the same dimensions, such that the second component can mate within the receiving compartment.

The term “comprising” includes, but is not limited to, whatever follows the word “comprising.” Use of the term indicates the listed elements are required or mandatory but that other elements are optional and may be present.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts or features that do not affect the basic operation of the apparatus, structure or method described.

The term “controlling fluid flow” or to “control fluid flow” refers to the ability to cause a fluid to move through a flow path in a specific direction, rate, or route.

The term “cut-out” refers to a removed portion of an otherwise continuous side or base of a component.

A “dialysate flow path” is a route in which a fluid can travel during dialysis.

“Dialysis” or “dialysis therapy” is a type of filtration, or a process of selective diffusion through a membrane. Dialysis removes solutes of a specific range of molecular weights via diffusion through a membrane from a fluid to be dialyzed into a dialysate. During dialysis, a fluid to be dialyzed is passed over a filter membrane, while dialysate is passed over the other side of that membrane. Dissolved solutes are transported across the filter membrane by diffusion between the fluids. The dialysate is used to remove solutes from the fluid to be dialyzed. The dialysate can also provide enrichment to the other fluid.

A “dialysis machine” is a system comprising a dialyzer, pumps, valves and fluid lines that is used to carry out a dialysis session.

The term “disposed” refers to a first component's placement on a second component.

The term “downstream” refers to a position of a first component in a flow path relative to a second component wherein fluid will pass by the second component prior to the first component during normal operation. The first component can be said to be “downstream” of the second component, while the second component is “upstream” of the first component.

To “extend” refers to a component spanning a defined area or direction.

The term “extending horizontally” makes reference to a feature, such as a wall or a side of geometric shape, that extends from a point or line in a direction in a horizontal plane of the point or line.

The term “extending outwardly” makes reference to a feature that extends in a direction horizontally, vertically, or a combination thereof, from a second feature or component.

A “fluid” is a liquid substance optionally having a combination of gas and liquid phases in the fluid. Notably, a liquid can therefore also have a mixture of gas and liquid phases of matter.

The term “fluid connector,” “fluidly connectable,” or “fluidly connected” refers to the ability to pass fluid, gas, or mixtures thereof from one point to another point. The two points can be within or between any one or more of compartments, modules, systems, and components, all of any type.

The term “fluid pump” or “pump” refers to any device that causes the movement of fluids or gases by applying suction or pressure.

The term “formed integrally” refers to two components that are permanently connected.

A “handle” can be a component on an infusate frame that is used to hold the infusate frame while moving the infusate frame.

An “infusate container” is a container adapted to contain one or more fluids for use in dialysis. The infusate container can at times hold dry chemicals that are later able to be reconstituted with a fluid to form a further useable fluid within the system.

An “infusate frame” is a component detachably removable from a dialysis system and having a substantially planar shape, configured to hold one or more containers.

A “ledge” is a flat structure extending outwardly from a side of a component.

The term “lip” refers to an edge portion of a component that contacts another component to result in a static position.

A “locked state” refers to a configuration of attached components wherein the components cannot easily be detached from one another.

A “locking mechanism” is any mechanism by which one component can be connected to a second component and resist inadvertent disconnection.

An “opening” is a portion of a component having a defined void space. As used in the invention, an opening in an infusate frame refers to a void space into which an infusate container can be inserted.

The term “opposedly positioned” refers to relative positions of two or more components wherein the two or more components are positioned on opposite sides of a reference.

The term “oval” refers to a two-dimensional shape having rounded ends and a slightly elongated shape.

The term “paddles” refers to components that can be rotatable, and in a preferred embodiment the paddles rotationally extend axially from a central axis. Multiple paddles can be used together as a “paddle assembly.”

The term “periphery” refers to an outer edge of a component.

The term “planar surface” refers to a surface of a component that is substantially flat and extending in two dimensions.

A “receiving compartment” is a portion of a container, caddy, device, or system adapted for receiving a component or container.

The term “removable” or “removed” relates to any component of the present invention that can be separated from a system, module, cartridge or any component of the invention.

The term “rigid” refers to a component having a substantially stiff structure that resist bending and is not generally flexible.

“Shape” refers to the three dimensional form of a component.

“Size” refers to the area, surface area, or volume of a container or component.

The term “slotted” refers to an opening extending inwardly from an outer edge of a component.

The terms “sodium bicarbonate container” refers to an object that can be a stand-alone enclosure or alternatively can be integrally formed with an apparatus for hemodialysis, hemodiafiltration, or hemofiltration. The object can store a source of sodium bicarbonate in solid and/or solution form, and can be configured to interface with at least one other functional module found in systems for hemodialysis, hemodiafiltration, or hemofiltration. The sodium bicarbonate reservoir or container can contain at least one fluid pathway and include components such as conduits, valves, filters or fluid connection ports.

The terms “sodium chloride container” refers to an object that can be a stand-alone enclosure or alternatively can be integrally formed with an apparatus for hemodialysis, hemodiafiltration, or hemofiltration. The object can store a source of sodium, such as sodium chloride in solid and/or solution form, and can be configured to interface with at least one other functional module found in systems for hemodialysis, hemodiafiltration, or hemofiltration. The sodium chloride reservoir or container can contain at least one fluid pathway and include components such as conduits, valves, filters or fluid connection ports.

A “solute” is a substance dissolved in, or intended to be dissolved in, a solvent.

The term “square” refers to a two-dimensional shape having four edges and four angles. This description is not intended to limit the size and dimensions of the described components, and may therefore encompass components having corners with angles greater than or less than ninety degrees, and with edges of differing lengths with respect to each other.

A “support member” or “support” is any structure connected to a component designed to hold or rigidly affix the component in a particular location, configuration, or orientation.

The term “tapered inwardly” refers to a three-dimensional part of a component that extends gradually towards a point when moving from the outside of the component to the inside of the component.

The term “triangular” refers to a two-dimensional shape having three sides.

The term “upstream” refers to a position of a first component in a flow path relative to a second component wherein fluid will pass by the first component prior to the second component during normal operation. The first component can be said to be “upstream” of the second component, while the second component is “downstream” of the first component.

The term “upwardly extending” makes reference to a feature, such as a wall or a side of geometric shape, that can be used to form a volume from a geometric based of any type. For example, a rectangular base having four sides can extend upwardly to form a cubic volume.

A “valve” is a device capable of directing the flow of fluid or gas by opening, closing or obstructing one or more pathways to allow the fluid or gas to travel in a path. One or more valves configured to accomplish a desired flow can be configured into a “valve assembly.”

The term “vertical axis” refers to an imaginary line extending upwardly and downwardly through a point.

### Infusate Frame

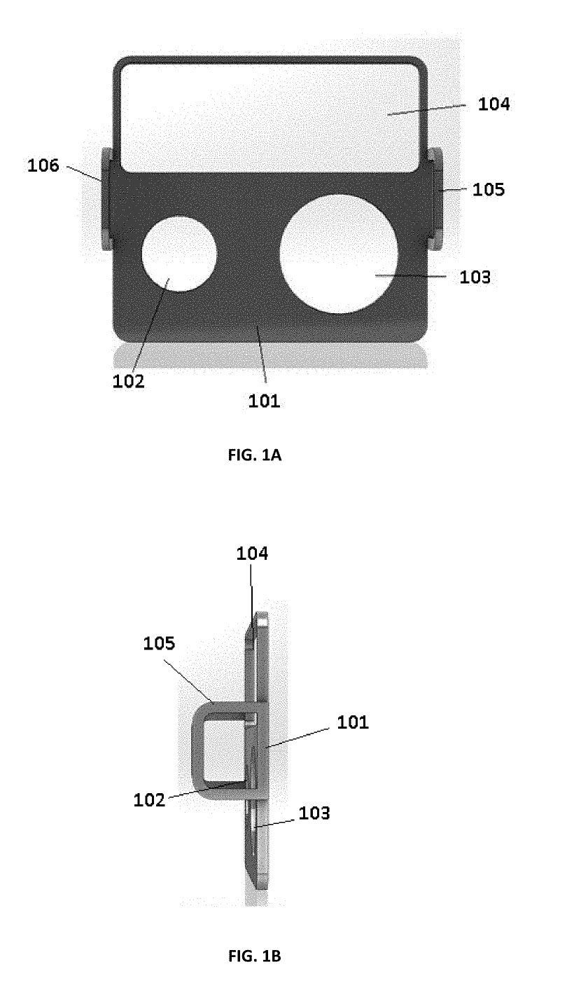

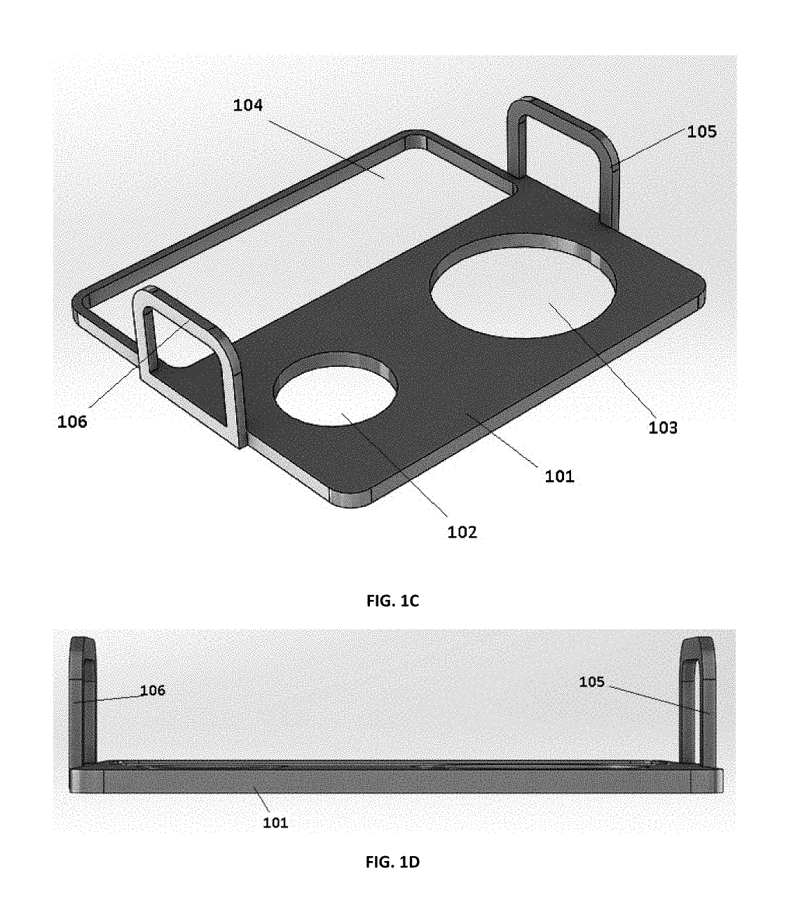

FIGS. 1A-D illustrate an infusate frame for containing one or more infusate containers. FIG. 1A is a top view of the infusate frame, FIG. 1B is a side view of the infusate frame, FIG. 1C is a perspective view of the infusate frame, and FIG. 1D is a front view of the infusate frame. The infusate frame can include a rigid planar surface 101 and one or more openings 102, 103, and 104 cut-out on the planar surface 101 sized to receive one or more infusate containers. Opening 102 can receive a sodium chloride container, opening 103 can receive a sodium bicarbonate container, and opening 104 can receive a cation infusate container. The infusate frame can include any number of openings for receiving any number of infusate containers, including 2, 3, 4, 5, 6, or more openings. The openings can be sized and shaped complementary to the infusate containers to ensure the proper infusate container is placed within the proper opening. A vertical axis of the openings 102, 103, and 104 can be aligned to one or more fluid connectors disposed on a dialysis machine, allowing each infusate container to only connect to the single fluid connector where the vertical axis of the opening is aligned with the fluid connector. The openings 102, 103, and 104 can extend from a first side to a second side of the planar surface 101, creating an opening through the entire planar surface 101. The edges of the planar surface 101 along the periphery can be a lip extending outwardly from the remainder of the planar surface 101. The lip can be disposed on the periphery of the planar surface 101 and support each side of the planar surface 101 when inserted into a receiving compartment (not shown) of a dialysis machine. The lip can be sized and shaped complementary to a ledge on the dialysis machine (not shown). When the infusate frame is inserted into the dialysis machine, the lip engages with the ledge to hold the infusate frame in place. The infusate containers can be inserted into the openings 102, 103, and 104 with the described container connectors extending through the openings 102, 103, and 104. When the fluid connectors are connected to the containers, the force is transferred to the planar surface 101. By transferring the force to the planar surface 101, a flexible container can be easily connected to the dialysis machine without deforming the flexible container. Any of the one or more openings in the planar surface 101 can be tapered inwardly, providing additional support for the infusate containers. The planar surface 101 of the infusate frame can also include handles 105 and 106 disposed on a periphery of the planar surface 101 for carrying and moving the infusate frame. The handles 105 and 106 can extend upwardly from the planar surface 101.

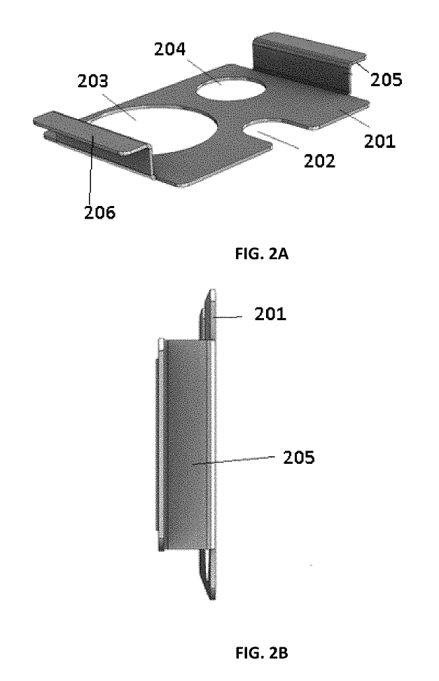

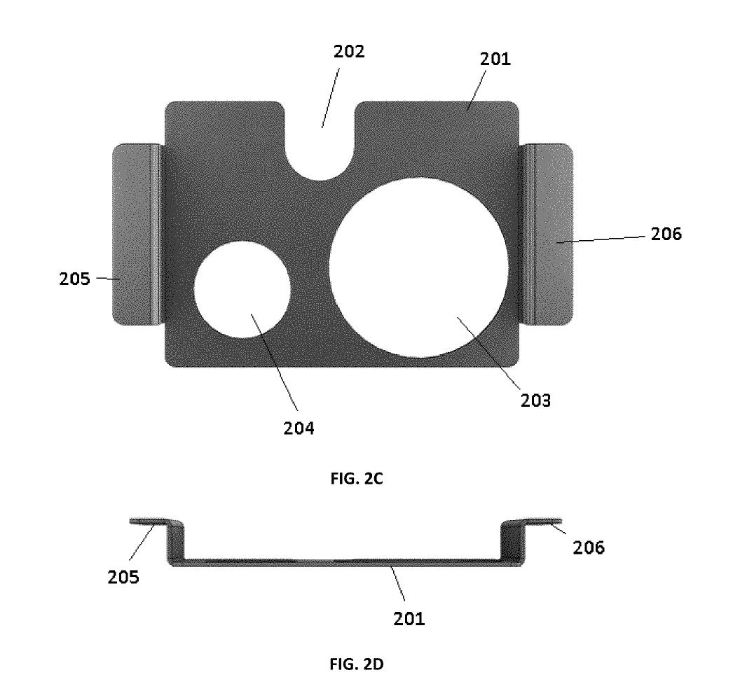

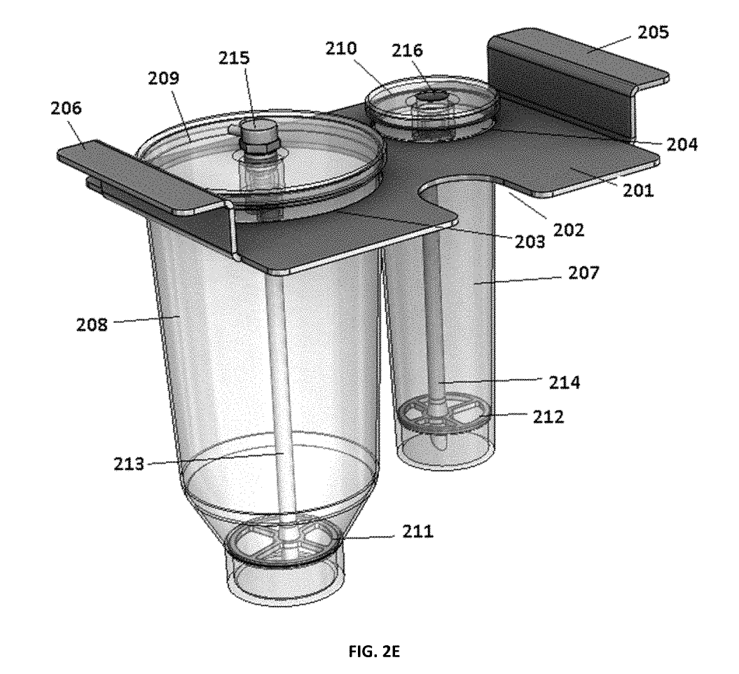

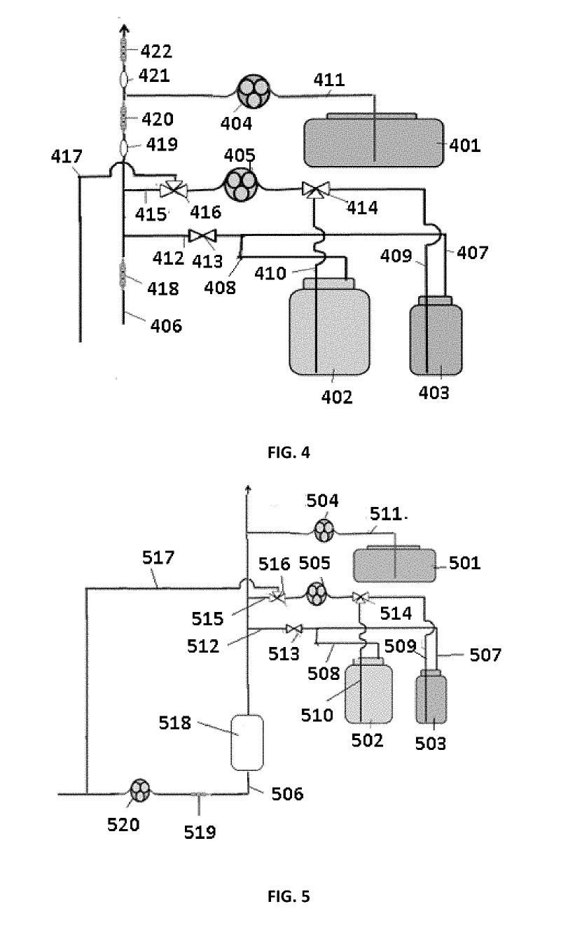

FIGS. 2A-E illustrates an infusate frame with a slotted opening 202 disposed on a periphery of the rigid planar surface 201. FIG. 2A is a perspective view of the infusate frame, FIG. 2B is a side view of the infusate frame, FIG. 2C is a top view of the infusate frame, FIG. 4D is a front view of the infusate frame, and FIG. 2E is a perspective view of the infusate frame with two infusate containers. The slotted opening 202 allows a flexible container to be inserted into the side of the planar surface 201 as opposed to from the top of the planar surface 201. The planar surface 201 can also include openings 203 and 204 cut-out from the planar surface 201 to receive one or more infusate containers. The slotted opening 202 as well as openings 203 and 204 can extend from a first side to a second side of the planar surface 201 to create an opening through the entirety of the planar surface 201. As illustrated in FIG. 2E, opening 203 can receive a sodium bicarbonate container 208, while opening 204 can receive a sodium chloride container 207. Because of the larger size of the sodium bicarbonate container 208 as compared to the smaller sodium chloride container 207, and the complementary sized openings 203 and 204, the sodium bicarbonate container can only be placed in the sodium bicarbonate opening 203 and the sodium chloride container 207 can only be placed in the sodium chloride opening 204. The slotted opening 202 can be sized and shaped complementary to a flexible or non-flexible infusate container (not shown). In FIG. 2E, the sodium bicarbonate container 208 is shown with a tapered bottom portion. As described, any of the containers can initially contain a solid infusate source. The inwardly tapering portion increases the efficiency of dissolving solid sodium bicarbonate within the sodium bicarbonate container 208. With the tapered bottom portion to the sodium bicarbonate container 208, the efficiency in dissolving and delivering sodium bicarbonate to the dialysis system is increased to greater than 90% when using a sodium bicarbonate container 208 with a tapered bottom portion, compared to about 50% when using a sodium bicarbonate container 208 without a tapered bottom portion. Due to the solubility, particle packing, and particle size of sodium chloride, the sodium chloride container 207 does not require a tapered bottom.

Lips 205 and 206, which are ledges disposed on a periphery of the planar surface 201 and outwardly extending from the planar surface 201, can be sized and shaped complementary to a ledge on a dialysis machine (not shown). The lips 205 and 206 can be opposedly positioned on opposite sides the planar surface 201. When the planar surface 201 is inserted into the dialysis machine, the lips 205 and 206 engage with the ledge to hold the infusate frame in place. The lips 205 and 206 can include vertical supports extending upwardly from the planar surface 201 with the lips 205 and 206 extending outwardly from the planar surface 201 horizontally from the vertical support, as illustrated in FIGS. 2A-E. Alternatively, the lips 205 and 206 can extend outwardly from the planar surface 201 in the same plane as the planar surface 201 without vertical supports. Further, the lips 205 and 206 can be the outer edges of the planar surface 201, as illustrated in FIGS. 1A-C.

The sodium chloride container 207, sodium bicarbonate container 208, and any additional infusate containers can be any type of infusate containers known in the art. As illustrated in FIG. 2E, the sodium chloride container 207 can have a removable cap 210 with connector 216 for connection to a dialysis machine, and the sodium bicarbonate container 208 can have a removable cap 209 including connector 215 for connection to a dialysis machine. The caps 210 and 209 can be removed to fill the infusate containers with the proper solutions and for cleaning of the containers. The infusate containers can hold either solid or liquid forms of the infusates. For example, sodium bicarbonate container 208 can contain solid sodium bicarbonate or an aqueous solution of sodium bicarbonate. When solid bicarbonate is used, the solid bicarbonate can be added to the sodium bicarbonate container 208 above support ledge 211. A filter or other separator can be placed on support ledge 211 to prevent the solid bicarbonate from moving into the bottom of the sodium bicarbonate container 211. Purified water from the dialysis machine can be flowed into the sodium bicarbonate container 208 through connector 215 to dissolve the solid sodium bicarbonate and make a solution of known concentration. The known concentration can be a saturated solution of sodium bicarbonate by adding more sodium bicarbonate into the sodium bicarbonate container 208 than can be dissolved in the added water. Alternatively, a known amount of sodium bicarbonate can be placed in sodium bicarbonate container 208 and a known amount of water added to create a sodium bicarbonate solution of known concentration. The sodium bicarbonate can be flowed from the sodium bicarbonate container 208, through draw tube 213 and connector 215 and into the dialysate flow path. Similarly, solid sodium chloride can be placed in sodium chloride container 207, with a filter on support ledge 212. Water can be added to the sodium chloride container 207 through connector 216 to dissolve the solid sodium chloride. The resulting sodium chloride solution can be flowed from the sodium chloride container 207 through draw tube 214 and connector 216 and into the dialysate flow path.

The infusate frame can be removable from the dialysis machine. Infusate containers can be placed in the infusate frame before or after inserting the infusate frame into the dialysis machine. Alternatively, the infusate frame can be non-removable, or formed integrally to the dialysis machine. The infusate containers can be placed into and removed from the infusate frame without removing the infusate frame from the dialysis machine.

After placing the infusate frame into a receiving compartment of the dialysis machine and placing the infusate containers into the infusate frame, the infusate containers can be connected to fluid connectors disposed on the dialysis machine. Each fluid connector can be aligned with a vertical axis of a single opening in the planar surface 201, ensuring that the correct fluid connector is connected to the correct infusate container during use. The fluid connectors can be any type of fluid connector known in the art. The fluid connectors can also be bi-directional connectors, allowing addition and removal of fluid from the infusate container through a single fluid connector.

In addition to having openings sized complementary to the infusate containers as illustrated in FIGS. 1-2, the infusate frame may having openings shaped complementary to the infusate containers. For example, an opening for a sodium chloride container can be circular shaped, while an opening for a sodium bicarbonate container can be rectangular shaped. The sodium chloride container can have a circular shape to correspond to the circular opening and the sodium bicarbonate container can have a rectangular shape to correspond to the rectangular opening. The openings and containers can have any shape, including a square shape, a circular shape, a rectangular shape, triangle shape, oval shape, or any other possible shape. Further, one or more visual indicators can be included to ensure the correct containers are placed in the correct opening of the infusate frame. For example, the infusate frame may be colored green around the sodium bicarbonate container opening and red around the sodium chloride container opening. The sodium bicarbonate container can also be green or include a green marking, and the sodium chloride container can be red or contain a red marking. The user need only match up the visual indicators on the containers to the visual indicators on the infusate frame to ensure the containers are in the proper locations in the infusate frame.

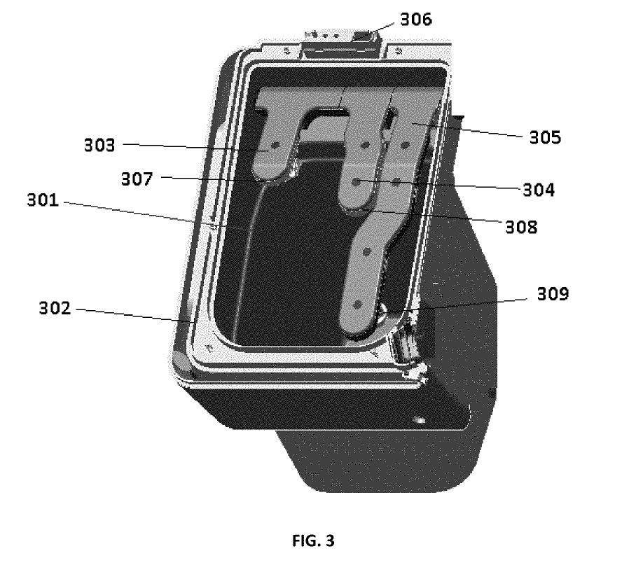

FIG. 3 illustrates a receiving compartment 301 of a dialysis machine for receiving an infusate frame. Ledge 302 can be sized and shaped complementary to the lip of the infusate frame. The infusate frame can be placed in the receiving compartment 301, with the infusate containers extending downwardly into the receiving compartment 301. When the infusate frame is inserted into the receiving compartment 301, the containers in the infusate frame will be aligned with fluid connectors 307, 308, and 309 on the dialysis machine because each opening in the infusate frame has a vertical axis aligned with a single connector. For example, fluid connector 307 can be aligned with the opening for the sodium bicarbonate container of the infusate frame, fluid connector 308 can be aligned with the opening for the sodium chloride container, and fluid connector 309 can be aligned with the opening for a cation infusate container.

The receiving compartment 301 can be sized and shaped complementary to the infusate frame with ledge 302 positioned complementary to the lip of the infusate frame. The receiving compartment 301, ledge 302, and infusate frame can be any shape, including rectangular shaped, circular shaped, oval shaped, triangle shaped, square shaped, or any other shape. Further, the infusate frame and receiving compartment 301 can be shaped to ensure that the infusate frame is only inserted in the proper orientation to align the infusate containers with the proper fluid connectors. For example, the receiving compartment 301 can have a smaller size or shape at one end and a larger size or shape on a second end. The infusate frame can be sized and shaped complementary, with different sizes or shaped on different ends of the infusate frame. The infusate frame will only be insertable into the receiving compartment 301 in a single orientation where the complementary sizes and shapes of the receiving compartment 301 and infusate frame match.

In addition to sizing and shaping the openings in the infusate frame to the infusate containers, an exterior surface of the fluid connectors can have a fitting feature to ensure proper mating to corresponding infusate container. For example, a first fluid connector can have a hexagonal-shape while a second fluid connector can have a circular-shape. The corresponding infusate containers can have surfaces matched to receive the hexagonal- or circular shaped fluid connectors. The fluid connectors and container connectors can have any complementary shape, including circular, rectangular, square, triangular, hexagonal, or any other shape known in the art. One skilled in the art will understand that additional solute containers can be included in the infusate frame, and that additional paddles and/or connectors can be included in the dialysis machine as necessary.

If FIG. 3, the fluid connectors 307, 308, and 309 are disposed on moveable paddles 303, 304, and 305, respectively. The paddles 303, 304, and 305 can be rotated about a hinge. The paddles 303, 304, and 305 can be moved upward to facilitate insertion of the infusate frame into the receiving compartment 301. After insertion, the paddles 303, 304, and 305 can be moved downward to place the fluid connectors 307, 308, and 309 in position to connect to each of the infusate containers. However, the fluid connectors 307, 308, and 309 need not be included on paddles, and can be included on a length of hose, wherein the hose is fluidly connected to a dialysate flow path. The hose can be made of any material known in the art for use in dialysis systems, including silicone, reinforced silicone, or PVC. One skilled in the art will understand that other biocompatible materials can be used for the hose, and the hose is not limited to any particular materials. The hoses can be either flexible or semi-rigid, which would allow the hoses to move for connection to the containers in the infusate frame. The hoses can be sized and positioned such that each hose will only be able to connect with a single container within the infusate frame. For example, each hose may be positioned on a specific location with respect to the receiving compartment 301, and each hose can be short enough so that the hose cannot reach any container not aligned with the specific location of the hose.

The receiving compartment 301 and infusate frame can also include an optional locking mechanism to keep the infusate frame from moving after insertion into the receiving compartment 301. Non-limiting examples of locking mechanisms include latches that engage when the infusate frame is inserted in the receiving compartment 301. The infusate frame cannot be moved once the latches are engaged until a user disengages the latches. The locking mechanism can be positionable in an open state, which will allow removal of the infusate frame or a locked state, which will prevent the infusate frame from moving. Any locking mechanism that can keep the infusate frame from moving within the receiving compartment 301 can be used.

FIG. 4 illustrates a non-limiting flow diagram for infusate containers seated in an infusate frame fluidly connected to a dialysate flow path 406. The infusate frame can contain a cation infusate container 401, a sodium bicarbonate container 402, and a sodium chloride container 403, each of which can contain a solid source or a concentrate. Because the openings cut-out in the infusate frame are sized or shaped complementary to the infusate containers, the infusate containers can only be attached to the specific fluid connectors aligned with the openings, ensuring that the proper chemicals are added to a dialysate flow path 406 at the proper locations. Additional containers can be included in the infusate frame as necessary. Sodium chloride container 403 can be connected to fluid lines 407 and 409. Fluid line 407 can connect the sodium chloride container 403 to valve 413. Valve 413 can also connect to fluid line 412, which in turn connects to the main dialysate flow path 406 allowing fluid from the dialysate flow path 406 to enter the sodium chloride container 403. Fluid line 409 can connect to valve 414, which also connects to valve 416 downstream of pump 405. Pump 405 can control fluid flow through fluid line 409 and valve 416, and can be a bidirectional pump. Valve 416 can be operated to direct fluid into the main dialysate flow path 406 during treatment by fluid line 415, or alternatively to direct fluid through fluid line 417 to a separate portion of the dialysate flow path 406. As described, directing sodium chloride and sodium bicarbonate upstream of a sorbent cartridge (not shown in FIG. 4) can reduce the time necessary for priming the dialysis machine. Valve 416 allows the sodium chloride and sodium bicarbonate to be pumped either upstream or downstream of the sorbent cartridge during priming and treatment, respectively.

Sodium bicarbonate container 402 can be connected to fluid lines 410 and 408. Fluid line 408 can also connect to valve 413 and can allow fluid from the dialysate flow path 406 to enter the sodium bicarbonate container 402. Fluid line 410 can also connect to valve 414. Cation infusate container 401 can be connected by fluid line 411 to the main dialysate flow path 406. Pump 404 can control fluid flow through line 411. Either or both of pumps 404 and 405 can be bi-directional pumps to move fluid from the containers within the infusate frame to the main dialysate flow path 406, or from the main dialysate flow path 406 to any of the containers within the infusate frame.

During treatment, various sensors can determine the concentration of sodium chloride, sodium bicarbonate, and cations added to the dialysate flow path 406 from the infusate frame containers. Conductivity sensor 418 can determine the conductivity of the dialysate prior to addition of sodium bicarbonate, sodium chloride, or other cations. Based on the conductivity detected by conductivity sensor 418, the amount of each fluid that needs to be added to the dialysate can be determined. Conductivity sensor 420, located downstream of fluid line 415, can determine the conductivity of the dialysate after addition of sodium bicarbonate, and ensures that the correct amount of sodium bicarbonate is added to the dialysate. Static mixer 419 can ensure complete mixing of the added sodium bicarbonate and the dialysate for accurate measurements by conductivity sensor 420. Conductivity sensor 422, located downstream of fluid line 411, can determine the conductivity of the dialysate after addition of the cation infusates, and ensure that the correct amount the cations is added to the dialysate. Conductivity sensor 422 can also provide a final check of the dialysate conductivity prior to the dialysate entering the dialyzer (not shown in FIG. 4). If the detected conductivity is outside of a predetermined range, the system can provide an alert, shutdown, or bypass the dialyzer to avoid delivering an unsafe dialysate to the patient. Static mixer 421 can ensure complete mixing of the added cation infusates and the dialysate for accurate measurements by conductivity sensor 422. One of skill in the art will understand that alternative arrangements of fluid lines, pumps, and valves are possible with the infusate frame and FIG. The static mixers and sensors illustrated in FIG. 4 can be included in any infusate frame configuration.

FIG. 5 illustrates a simplified portion of a dialysate flow path 506 using the infusate frame flow diagram. Sodium chloride container 503 can be connected to fluid lines 507 and 509. Fluid line 507 can connect the sodium chloride container 503 to valve 513. Valve 513 can also connect to fluid line 512, which in turn connects to the main dialysate flow path 506 allowing water from the dialysate flow path 506 to enter the sodium chloride container 503 for priming of the sodium chloride container 503. Fluid line 509 can connect to valve 514, which also connects to valve 516 downstream of pump 505. Pump 505 can control fluid flow through line 509 and valve 516. Valve 516 can be selectively openend and closed to direct fluid into the main dialysate flow path 506 during treatment by fluid line 515, or alternatively to direct fluid through fluid line 517 to a separate portion of the dialysate flow path 506.

Sodium bicarbonate container 502 can be connected to fluid lines 510 and 508. Fluid line 508 can also connect to valve 513 and can allow water from the dialysate flow path 506 to enter the sodium bicarbonate container 502. Fluid line 510 can also connect to valve 514. Cation infusate container 501 can be connected by fluid line 511 to the main dialysate flow path 506. Pump 504 can control fluid flow through line 511.

As described, valve 516 allows fluid to be directed to the dialysate flow path 506 upstream of sorbent cartridge 518. To reuse a dialyzer (not shown in FIG. 15), the dialyzer can be sterilized with a disinfectant solution. The disinfectant solution must then be flushed out of the dialyzer and dialysate flow path 506 by pumping fluid through the dialysate flow path 506. The sorbent cartridge 518 must then be flushed, drained, conditioned with sodium bicarbonate and primed with sodium chloride. Without valve 516, sorbent cartridge 518 can fill with water prior to conditioning. The sorbent cartridge 518 can then be flushed with additional sodium bicarbonate solution that has passed through the entire dialysate flow path 506 for conditioning. By directing the sodium bicarbonte through fluid line 517 upstream of the sorbent cartridge 518, only fluid with sodium bicarbonate enters the sorbent cartridge 518, reducing the time necessary for conditioning of the sorbent cartridge 518. After conditioning, the sorbent cartridge 518 is primed with a sodium chloride solution. By directing the sodium chloride through fluid line 517 upstream of the sorbent cartridge 518, only fluid with sodium sodium chloride enters the sorbent cartridge 518, reducing the time necessary for priming of the sorbent cartridge 518. The total time for conditioning and priming the system can be reduced by as much as 5-15 minutes by directing fluid upstream of the sorbent cartridge 518 with valve 516. Pump 520 provides the driving force for conveying dialysate and priming solution through the dialysate flow path 506. Conductivity sensor 519 detects the conductivity of the fluid prior to entering the sorbent cartridge 518. The conductivity of the fluid, along with the flow rate of the fluid, can be used determine an amount of bicarbonate and sodium chloride pumped through the dialysate flow path 506, allowing closed loop control during priming and flushing. One of skill in the art will understand a valve similar in function to valve 516 can be included in any of the described infusate frame configurations.

One skilled in the art will understand that various combinations and/or modifications and variations can be made in the described systems and methods depending upon the specific needs for operation. Moreover features illustrated or described as being part of an aspect of the invention may be used in the aspect of the invention, either alone or in combination.

We claim:

1. An infusate frame, comprising:
a rigid planar surface having at least a first lip disposed at a periphery of the planar surface and extending outwardly from the planar surface; the lip complementary to a ledge disposed on a dialysis machine; and
one or more openings cut-out on the planar surface; the one or more openings extending from a first side to a second side of the planar surface; the one or more openings having a size and/or shape complementary to one or more infusate container;
wherein a vertical axis of the one or more openings is aligned to a fluid connector disposed on the dialysis machine.

2. The infusate frame of claim 1, wherein the one or more openings are tapered inwardly.

3. The infusate frame of claim 1, wherein at least one opening is slotted to a periphery of the planar surface.

4. The infusate frame of claim 3, wherein the one or more openings are selected from a circular shape, a square shape, a triangular shape, or an oval shape.

5. The infusate frame of claim 1, further comprising one or more handles disposed on a periphery of the planar surface; the one or more handles extending upwardly from the planar surface.

6. The infusate frame of claim 1, wherein the one or more infusate container is selected from the group consisting of a sodium bicarbonate container, a sodium chloride container, and a cation infusate container.

7. The infusate frame of claim 1, further comprising at least one vertical support disposed on a periphery of the planar surface; the at least one vertical support extending upwardly from the periphery of the planar surface with a lip extending horizontally from the at least one vertical support.

8. The infusate frame of claim 1, comprising at least two openings cut-out on the planar surface, the at least two openings having different sizes or shapes.

9. The infusate frame of claim 1, further comprising a second lip opposedly positioned on the planar surface to the first lip.

10. A dialysis system comprising:
the infusate frame of claim 1, having one or more infusate containers containing one or more solute;
a dialysis machine, comprising:
(i) a dialysate flow path;
(ii) a receiving compartment for the infusate frame; and
(iii) one or more fluid connectors fluidly connectable to the one or more infusate containers and the dialysate flow path.

11. The dialysis system of claim 10, further comprising one or more pumps and one or more valves fluidly connectable to the one or more fluid connectors; the one or more pumps and one or more valves controlling fluid flow from the one or more infusate containers into the one or more fluid connectors.

12. The dialysis system of claim 10, further comprising a locking mechanism preventing the infusate frame from moving after insertion into the receiving compartment when the locking mechanism is in a locked state.

13. The dialysis system of claim 10, wherein the receiving compartment is sized or shaped complementary to a size or shape of the infusate frame.

14. The dialysis system of claim 10, wherein the infusate frame is formed integrally with the dialysis machine.

15. The dialysis system of claim 10, wherein the infusate frame is removable from the dialysis machine.

16. The dialysis system of claim 11, wherein at least one pump is a bidirectional pump.

17. The dialysis system of claim 10, wherein at least one fluid connector is disposed on a moveable paddle.

18. The dialysis system of claim 10, wherein the one or more infusate containers include a cation infusate container, a sodium chloride container, a sodium bicarbonate container, or combinations thereof.

19. The dialysis system of claim 18, wherein the sodium chloride container and sodium bicarbonate container are fluidly connectable to a valve; wherein the valve is fluidly connected to the dialysate flow path upstream of a sorbent cartridge and downstream of the sorbent cartridge.

20. The dialysis system of claim 10, wherein at least one fluid connector is a bi-directional connector.
